(19) United States
(12) Patent Application Publication
(10) Pub. No.: US 2022/0262497 A1
(43) Pub. Date: Aug. 18, 2022

(54) NURSING ASSISTANCE DEVICE

(71) Applicant: FIRST ASCENT INC., Tokyo (JP)

(72) Inventor: Tomoyuki HATTORI, Tokyo (JP)

(73) Assignee: FIRST ASCENT INC., Tokyo (JP)

(21) Appl. No.: 17/608,354

(22) PCT Filed: May 17, 2019
(86) PCT No.: PCT/JP2019/019706

§ 371 Date: Nov. 2, 2021

(51) Int. Cl. G16H 20/70; H04M 11/00; A61B 5/374

(52) U.S. Cl. CPC G16H 20/70; A61B 5/374; H04M 11/00

(57) ABSTRACT

A nursing assistance device includes: a biological information acquisition unit that acquires biological information of an infant/toddler; a biological information estimation unit that, by using the biological information in a first period as acquired by the biological information acquisition unit and reference information based on the biological information of a plurality of infants/toddlers, estimates the biological information in a second period different from the first period; and an assessment unit that assesses an emotional abnormality in the infant/toddler in the second period on the basis of a comparison between the biological information in the second period as acquired by the biological information acquisition unit and the biological information as estimated by the biological information estimation unit.

### TECHNICAL FIELD

The present invention relates to a childcare (nursing) assistance device.

### BACKGROUND ART

In Patent Document 1, an infant monitoring system is disclosed which is capable of estimating an emotional state of an infant, such as whether the infant is receiving stress or the like, from measurement data related to the infant, on the basis of an environmental suitability model.

### PRIOR ART DOCUMENTS

### Patent Literature

Patent Document 1: International Patent Application Publication No. WO 2016/164373 A1

### SUMMARY OF INVENTION

### Technical Problem

The environmental suitability model used in the infant monitoring system disclosed in Patent Document 1 is based on empirical recognition, and a problem arises in that the accuracy of an estimation result concerning the emotional state of the infant including stress that is performed based on such an environmental suitability model is dependent on the accuracy of the aforementioned empirical recognition.

### Solution to Problem

According to a first aspect of the present invention, a childcare assistance device comprises a biological information acquisition unit configured to acquire biological information of an infant, a biological information estimation unit configured to estimate, using the biological information in a first period acquired by the biological information acquisition unit, and reference information based on the biological information of a plurality of infants, the biological information in a second period that differs from the first period, and a determination unit configured to determine an emotional abnormality of the infant in the second period, on a basis of a comparison between the biological information in the second period acquired by the biological information acquisition unit, and the biological information estimated by the biological information estimation unit.

According to a second aspect of the present invention, in the childcare assistance device according to the first aspect, the biological information acquisition unit preferably acquires biological information of the plurality of infants, and the reference information is based on accumulated information that includes the biological information of the plurality of infants acquired by the biological information acquisition unit.

According to a third aspect of the present invention, in the childcare assistance device according to the first aspect or the second aspect, the biological information estimation unit preferably estimates the biological information in the second period, using: the biological information in the first period acquired by the biological information acquisition unit; the biological information in a third period acquired by the biological information acquisition unit, the third period differing from both the first period and the second period; and the reference information.

According to a fourth aspect of the present invention, in the childcare assistance device according to any one of the first aspect to the third aspect, there is preferably further provided a behavior information acquisition unit configured to acquire behavior information indicating a behavior of the infant in the first period in which the biological information is measured, and to associate the behavior information with the biological information in the first period acquired by the biological information acquisition unit. The biological information estimation unit estimates the biological information in the second period for each of types of the behavior information, using the biological information in the first period with which the behavior information is associated by the behavior information acquisition unit, and the reference information which is based on the biological information in the first period and corresponds to the types of the behavior information. The determination unit determines the emotional abnormality of the infant in the second period and the behavior of the infant in the second period, on a basis of the comparison between the biological information in the second period acquired by the biological information acquisition unit, and the biological information in the second period estimated by the biological information estimation unit for each of the types of the behavior information.

According to a fifth aspect of the present invention, in the childcare assistance device according to the fourth aspect, there is preferably further provided an emotional information acquisition unit configured to acquire emotional information indicating whether or not the emotional abnormality has occurred in the infant during the behavior in the first period in which the biological information is measured, and to associate the emotional information with the biological information in the first period acquired by the biological information acquisition unit. The biological information estimation unit estimates the biological information in the second period for each of combinations of a type of the behavior information and a presence or absence of occurrence of the emotional abnormality, using the biological information in the first period with which the emotional information is associated by the emotional information acquisition unit, and the reference information which is based on the biological information of the plurality of infants and corresponds to the types of the behavior information and the presence or absence of the occurrence of the emotional abnormality. The determination unit determines the behavior and the emotional abnormality of the infant in the second period, on a basis of the comparison between the biological information in the second period acquired by the biological information acquisition unit, and the biological information in the second period estimated by the biological information estimation unit for each of the combinations.

According to a sixth aspect of the present invention, in the childcare assistance device according to the fourth or the fifth aspect, there are preferably further provided a caregiver information acquisition unit configured to acquire caregiver information indicating a caregiver of the infant when the biological information acquired by the biological information acquisition unit is measured, a childcare record acquisition unit configured to acquire a childcare record of the infant created by the caregiver, and a childcare record inspection unit configured to inspect the childcare record acquired by the childcare record acquisition unit, by comparing the childcare record with the behavior of the infant in the second period as determined by the determination unit.

According to a seventh aspect of the present invention, in the childcare assistance device according to any one of the first aspect to the third aspect, there is preferably further provided a caregiver information acquisition unit configured to acquire caregiver information indicating a caregiver of the infant when the biological information acquired by the biological information acquisition unit is measured. The determination unit determines whether or not the caregiver is suitable to provide childcare for the infant, on a basis of a result of determination of the emotional abnormality of the infant in the second period.

According to an eighth aspect of the present invention, in the childcare assistance device according to the seventh aspect, the determination unit preferably determines whether or not the caregiver is suitable to provide childcare for another infant that differs from the infant, on a basis of a result of determination as to whether or not the caregiver of the infant in the second period is suitable to provide childcare for the infant.

According to a ninth aspect of the present invention, in the childcare assistance device according to any one of the first aspect to the eighth aspect, there is preferably further provided a notification information generating unit configured to generate notification information in order to notify, via an electronic device, a caregiver of the infant in the second period or a manager of the caregiver that the emotional abnormality of the infant has been determined by the determination unit.

### Advantageous Effects of Invention

According to the present invention, an effect is obtained in that it is highly possible that emotional abnormalities of various infants can be appropriately determined for each infant.

### DESCRIPTION OF DRAWINGS

FIG. 1 is a diagram showing an exemplary configuration of a childcare assistance system including a childcare assistance device according to an embodiment of the present invention and a brain wave sensor, and an example of using the brain wave sensor;

FIG. 2 is a diagram showing a configuration of the childcare assistance device according to the embodiment;

FIG. 3 is a diagram illustrating a manner in which brain wave data is acquired as biological information of an infant;

FIG. 4 is a diagram showing an example of reference information that is generated based on brain wave data of a plurality of infants;

FIG. 5 is a diagram for explaining a processing example of estimating recent or current brain wave data, using brain wave data of a specified infant measured in the past;

FIG. 6 is a diagram illustrating a determination result of a behavior and an emotional abnormality of a specified infant, and a display screen of an electronic device that has received notification information for notifying a babysitter of the determination result;

FIG. 7 is a diagram for describing an example of a reference information generation process and an infant behavior and emotional abnormality determination process executed by running a computer program in the childcare assistance device according to the embodiment;

FIG. 8 is a diagram illustrating a product supplying mode of a computer program that runs in the childcare assistance device according to the embodiment;

FIG. 9 is a diagram for describing an example of a babysitter evaluation process executed by a childcare assistance device in an Exemplary Modification 4 of the embodiment of the present invention;

FIG. 10 is a diagram for describing an example of a babysitter evaluation process executed by a childcare assistance device in an Exemplary Modification 5 of the embodiment of the present invention;

FIG. 11 is a diagram for describing an example of a babysitter childcare record inspection process executed by a childcare assistance device in an Exemplary Modification 6 of the embodiment of the present invention; and

FIG. 12 is a diagram showing vocalization data acquired as biological information of an infant by a childcare assistance device in an Exemplary Modification 7 of the embodiment of the present invention.

### DESCRIPTION OF EMBODIMENTS

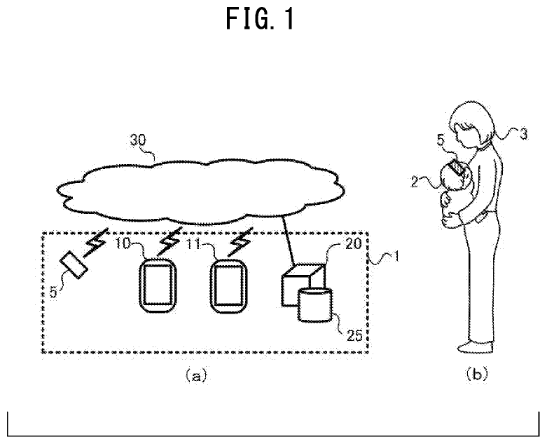

FIG. 1 is a diagram showing an exemplary configuration of a childcare assistance system 1 including a childcare assistance device 20 according to an embodiment of the present invention and a brain wave sensor 5, and an example of using the brain wave sensor 5. As illustrated in FIG. 1(a), the childcare assistance system 1 includes the brain wave sensor 5 that measures brain wave data of an infant 2, an electronic device 10 possessed by a parent or another guardian who provides childcare for the infant 2, an electronic device 11 possessed by a babysitter 3 who temporarily provides childcare for the infant 2 or a manager of the babysitter 3, the childcare assistance device 20 to which the brain wave sensor 5, the electronic device 10, and the electronic device 11 are connected via a communication network 30, and a storage device 25. In the present embodiment, the storage device 25 is externally connected to the childcare assistance device 20, but the storage device 25 may be incorporated internally in the childcare assistance device 20. As illustrated in FIG. 1(b), when the babysitter 3 temporarily provides childcare for the infant 2 during an interval in which the parent or another guardian is absent, the infant 2 wears the brain wave sensor 5, whereby the brain wave data can be measured as biological information of the infant 2.

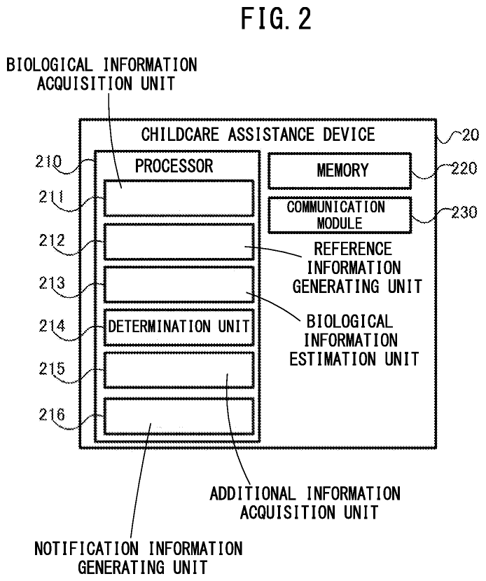

FIG. 2 is a diagram showing a configuration of the childcare assistance device 20 according to the present embodiment. The childcare assistance device 20 includes a processor 210, a memory 220, and a communication module 230. By activating a computer program stored in the memory 220, the processor 210 of the childcare assistance device 20 logically includes a biological information acquisition unit 211, a reference information generating unit 212, a biological information estimation unit 213, a determination unit 214, an additional information acquisition unit 215, and a notification information generating unit 216.

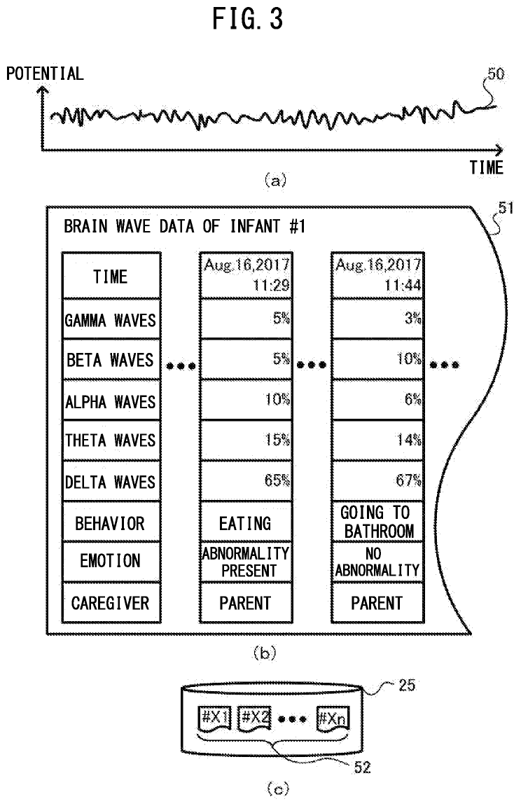

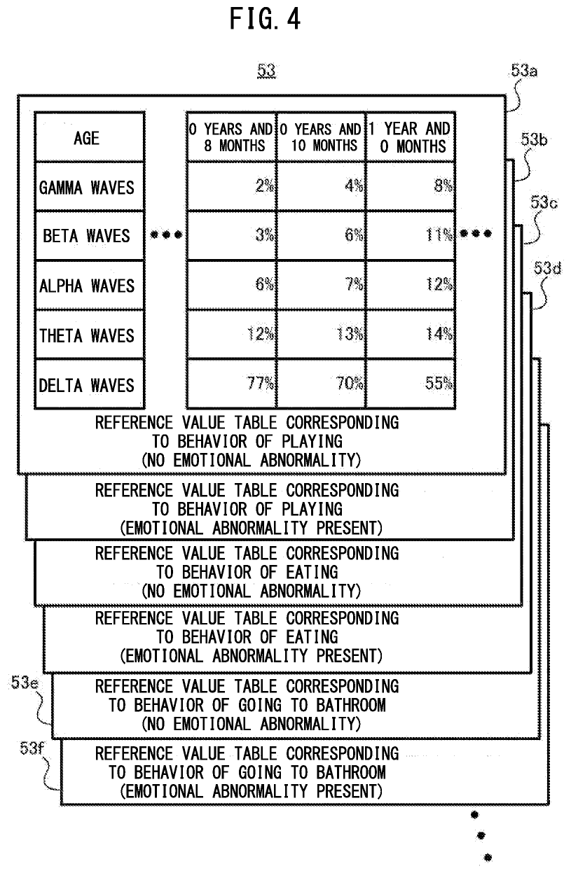

The biological information acquisition unit 211 acquires, via the communication module 230, brain wave data of the infant 2, which is measured by the brain wave sensor 5 as biological information of the infant 2, and stores the brain wave data in the storage device 25. Similarly, the biological information acquisition unit 211 also acquires brain wave data of other infants, and stores such brain wave data in the storage device 25. In this manner, brain wave data of a plurality of infants, which have been measured in the past by the brain wave sensor 5 and acquired by the biological information acquisition unit 211, are stored in the storage device 25. The brain wave data stored in the storage device 25 includes brain wave data of the infant 2 which is measured in a past period Tp1 by the brain wave sensor 5 and acquired by the biological information acquisition unit 211, brain wave data of the infant 2 which is measured by the brain wave sensor 5 in a past period Tp2 that differs from the period Tp1 and acquired by the biological information acquisition unit 211, and brain wave data of other infants which are measured in a past period Tp3 by the brain wave sensor 5 and acquired by the biological information acquisition unit 211. At least a portion of the brain wave data of the plurality of infants stored in the storage device 25 is the accumulated information shown in FIG. 3(c), to be described later. The reference information generating unit 212 generates reference information, which will be described later with reference to FIG. 4, based on the accumulated information stored in the storage device 25.

The biological information estimation unit 213 estimates the brain wave data of the infant 2 in a recent or current period Tc, using the brain wave data of the infant 2, which is measured by the brain wave sensor 5 in the past period Tp1 and in the past period Tp2 that differs from the period Tp1, and is acquired by the biological information acquisition unit 211, and the reference information generated by the reference information generating unit 212.

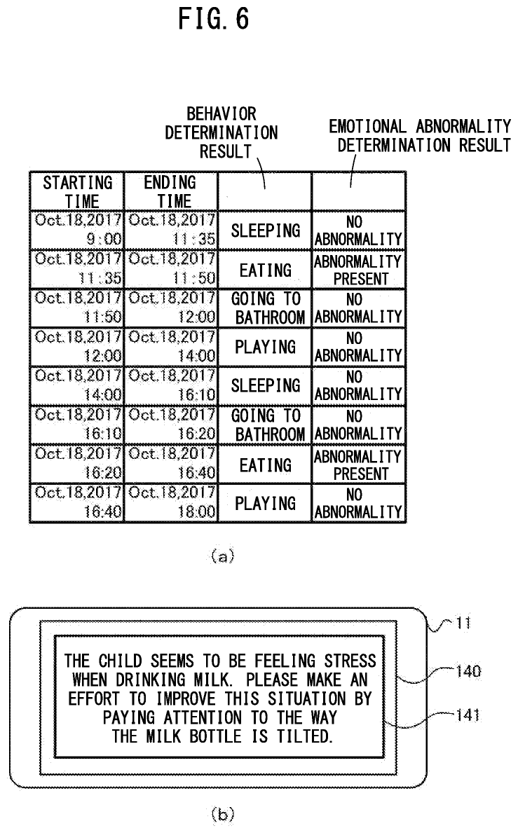

When the biological information acquisition unit 211 actually acquires the brain wave data of the infant 2 in the aforementioned period Tc, the determination unit 214 determines a behavior and an emotional abnormality of the infant 2 (described later with reference to FIG. 6) in the period Tc, based on the comparison between the brain wave data of the infant 2 acquired in such a period Tc, and the brain wave data of the infant 2 in the period Tc estimated by the biological information estimation unit 213.

It is assumed that the biological information acquisition unit 211 acquires the brain wave data of the infant 2, which is measured by the brain wave sensor 5 in the past period Tp1 and the past period Tp2 in which a parent (or another guardian) of the infant 2 has provided childcare for the infant 2, and such data is stored in the storage device 25. Thereafter, the parent of the infant 2 can input, into the electronic device 10, behavior information indicating behaviors of the infant 2 in the past period Tp1 and the past period Tp2 in which brain wave data of the infant 2 was measured, emotional information indicating whether or not an emotional abnormality occurred in the infant 2 during such behaviors, and caregiver information indicating a caregiver of the infant 2 during such behaviors. When the input behavior information, emotional information, and caregiver information are transmitted from the electronic device 10 to the childcare assistance device 20, the additional information acquisition unit 215 acquires the behavior information, the emotional information, and the caregiver information for the infant 2 via the communication module 230. The additional information acquisition unit 215 associates the acquired behavior information, emotional information, and caregiver information for the infant 2 with the brain wave data of the infant 2 which is measured by the brain wave sensor 5 in the past period Tp1 and the past period Tp2 and stored in the storage device 25.

By associating the behavior information and the emotional information of the infant 2 with the brain wave data of the infant 2, the reference information generating unit 212 is capable of generating, as the aforementioned reference information, a plurality of reference value tables corresponding to the types of behavior information and the presence or absence of the occurrence of an emotional abnormality. By associating the caregiver information of the infant 2 with the brain wave data of the infant 2, the reference information generating unit 212 is capable of generating the above-described reference information, on the basis of the brain wave data of the infant 2 during a period in which the caregiver of the infant 2 is a parent.

The notification information generating unit 216 generates notification information for notifying the parent of the infant 2 and the babysitter 3 that a behavior and an emotional abnormality of the infant 2 in the period Tc have been determined by the determination unit 214. The notification information generating unit 216 can transmit the notification information via the communication module 230 to the electronic device 10 that is possessed by the parent of the infant 2 and the electronic device 11 that is possessed by the babysitter 3, and cause the notification information to be displayed thereon. Moreover, in the case that a manager of the babysitter 3 possesses the aforementioned electronic device 11, the notification information is transmitted to the manager of the babysitter 3.

FIG. 3 is a diagram illustrating a manner in which brain wave data 51 is acquired as biological information of the infant 2. As illustrated in FIG. 3(a), brain waves 50 of the infant 2 which are measured by the brain wave sensor 5 are represented by a waveform for which the potential is represented on the vertical axis, and time is represented on the horizontal axis. In the brain waves 50, there are included various types of brain waves, and more specifically, a plurality of frequency components such as gamma waves, beta waves, alpha waves, theta waves, and delta waves are included therein.

It is assumed that an infant 2 #1, who is currently 0 years and 8 months old as of Aug. 16, 2017, was made to wear the brain wave sensor 5 for the entire day. The brain waves 50 of the infant 2 #1 are measured every minute by the brain wave sensor 5, and a measurement result thereof is acquired, as data that makes up a portion of the brain wave data 51 illustrated in FIG. 3(b), by the biological information acquisition unit 211 included in the processor 210 of the childcare assistance device 20. The brain wave data 51 illustrated in FIG. 3(b) includes frequency component percentages of the plurality of frequency components such as gamma waves, beta waves, alpha waves, theta waves, and delta waves included within the brain waves 50 of the infant 2 #1.

In the example shown in FIG. 3(b), the respective frequency component percentages of the gamma waves, the beta waves, the alpha waves, the theta waves, and the delta waves at 11:29 on Aug. 16, 2017 are 5%, 5%, 10%, 15%, and 65%, respectively. With the record at 11:29 on Aug. 16, 2017 that makes up the brain wave data 51, there are also associated the behavior information, the emotional information, and the caregiver information at that time, which are acquired by the additional information acquisition unit 215 included in the processor 210 of the childcare assistance device 20. In accordance with the behavior information, the emotional information, and the caregiver information, the behavior of the infant 2 #1 at that time is eating, and there is an emotional abnormality, while in addition, the caregiver of the infant 2 #1 at that time is a parent of the infant 2 #1. The respective frequency component percentages of the gamma waves, the beta waves, the alpha waves, the theta waves, and the delta waves at 11:44 on Aug. 16, 2017 are 3%, 10%, 6%, 14%, and 67%, respectively. In accordance with the behavior information, the emotional information, and the caregiver information associated with the record at 11:44 on Aug. 16, 2017 that makes up the brain wave data 51, the behavior of the infant 2 #1 at that time is going to the bathroom (expelling urine or feces), and there is not an emotional abnormality, while in addition, the caregiver of the infant 2 #1 at that time is a parent of the infant 2 #1. In this manner, not only is the brain wave data 51 of the infant 2 #1 in a period in which the age of the infant is 0 years and 8 months acquired, but also the brain wave data 51 of the infant 2 #1 in various periods that have elapsed from the birth of the infant, such as a period in which the age of the infant is 0 years and 6 months and the like, is acquired, and such data is stored in the storage device 25 as discussed previously.

Similarly, brain wave data of a plurality of infants 2 #X1, #X2, □, #Xn are obtained in various periods that have elapsed from the births of the respective infants, and as shown in FIG. 3(c), accumulated information 52 in which brain wave data of the plurality of infants 2 is included is stored in the storage device 25. It should be noted that it makes no difference as to whether or not the brain wave data 51 of the infant 2 #1 is included within the accumulated information 52.

FIG. 4 is a diagram showing an example of reference information 53 that is generated based on brain wave data of the plurality of infants 2. Since the frequency component percentages of the plurality of frequency components included within the brain waves of the infants 2 vary according to the age of the infants 2, the reference information 53 includes, for each age group, the frequency component percentages that make up the brain waves as shown in FIG. 4. The reference information 53 includes a plurality of reference value tables corresponding to the types of behavior information of the infants 2 and the presence or absence of the occurrence of an emotional abnormality. As shown in FIG. 4, the reference information 53 includes, for example, a reference value table 53a corresponding to a behavior in which the infants 2 are playing with no emotional abnormality, a reference value table 53b corresponding to a behavior in which the infants 2 are playing with an emotional abnormality, a reference value table 53c corresponding to a behavior in which the infants 2 are eating with no emotional abnormality, a reference value table 53d corresponding to a behavior in which the infants 2 are eating with an emotional abnormality, a reference value table 53e corresponding to a behavior in which the infants 2 are going to the bathroom with no emotional abnormality, and a reference value table 53f corresponding to a behavior in which the infants 2 are going to the bathroom with an emotional abnormality. Moreover, in FIG. 4, one reference value table is generated for each of combinations of the type of behavior information of the infants 2 and the presence or absence of the occurrence of an emotional abnormality. However, taking into consideration differences in the characteristics of the plurality of infants 2, a plurality of reference value tables may be generated for each of combinations of the type of behavior information of the infants 2 and the presence or absence of the occurrence of an emotional abnormality. Further, as will be discussed later in relation to an Exemplary Modification 1, regardless of the presence or absence of the occurrence of an emotional abnormality, one or a plurality of the reference value tables may be generated for each of the types of behavior information of the infants 2.

As discussed previously, the reference information generating unit 212 included in the processor 210 of the childcare assistance device 20 generates the reference information 53 based on the accumulated information 52 that is stored in the storage device 25. For example, the reference value table 53a corresponding to a behavior in which the infants 2 are playing with no emotional abnormality is generated by the following procedure. The reference information generating unit 212 extracts, from the accumulated information 52, the brain wave data obtained in a state in which the plurality of infants 2 having an age of 1 year and 0 months are playing and there is no emotional abnormality, and calculates a statistical value, for example such as an average value, of the respective frequency component percentages. In addition, it is assumed that all of the plurality of infants 2 from which the extracted brain wave data is measured are cared for by their own parents during the period in which the brain wave data is measured. As a result of calculating the average value of the respective frequency component percentages in this manner, the frequency component percentages of the plurality of frequency components included within the brain waves 50 of the infants 2 in a period in which their age is 1 year and 0 months can be obtained as the reference value table 53a corresponding to a behavior in which the infants 2 are playing with no emotional abnormality.

In the example of the reference value table 53a shown in FIG. 4, the gamma waves, the beta waves, the alpha waves, the theta waves, and the delta waves that make up the brain waves 50, which are measured during a behavior in which the infants 2 are playing and there is no emotional abnormality in a period in which their age is 1 year and 0 months, are 8%, 11%, 12%, 14%, and 55%, respectively. The gamma waves, the beta waves, the alpha waves, the theta waves, and the delta waves that make up the brain waves 50, which are measured during a behavior in which the infants 2 are playing and there is no emotional abnormality in a period in which their age is 0 years and 10 months, are 4%, 6%, 7%, 13%, and 70%, respectively. The gamma waves, the beta waves, the alpha waves, the theta waves, and the delta waves that make up the brain waves 50, which are measured during a behavior in which the infants 2 are playing and there is no emotional abnormality in a period in which their age is 0 years and 8 months, are 2%, 3%, 6%, 12%, and 77%, respectively. Although in FIG. 4, illustration of all of them is omitted, in the reference value table 53a, there are included the respective frequency component percentages that make up the brain waves 50 that are measured during a behavior in which the infants 2 are playing and there is no emotional abnormality in various periods that have elapsed from the births of the infants 2.

Similarly, based on the brain waves 50 measured during a behavior in which the plurality of infants 2 are playing and there is an emotional abnormality in periods in which the ages of the infants are 1 year and 0 months, 0 years and 10 months, 0 years and 8 months, and various other periods, the reference value table 53b corresponding to a behavior in which the infants 2 are playing with an emotional abnormality is generated. Further, based on the brain waves 50 measured in periods in which the ages of the infants are 1 year and 0 months, 0 years and 10 months, 0 years and 8 months, and various other periods, and during a behavior in which the plurality of infants 2 are eating and when there is no emotional abnormality and when there is an emotional abnormality, respectively, the reference value table 53c corresponding to a behavior in which the infants 2 are eating with no emotional abnormality, and the reference value table 53d corresponding to a behavior in which the infants 2 are eating with an emotional abnormality are generated. Further, based on the brain waves 50 measured in periods in which the ages of the infants are 1 year and 0 months, 0 years and 10 months, 0 years and 8 months, and various other periods, and during a behavior in which the plurality of infants 2 are going to the bathroom and when there is no emotional abnormality and when there is an emotional abnormality, respectively, the reference value table 53e corresponding to a behavior in which the infants 2 are going to the bathroom with no emotional abnormality, and the reference value table 53f corresponding to a behavior in which the infants 2 are going to the bathroom with an emotional abnormality are generated. Furthermore, other reference value tables may be generated, respectively, based on the brain waves 50 measured in various periods that have elapsed from the births of the respective infants 2, and while the infants 2 are engaged in various other behaviors, and when there is no emotional abnormality and when there is an emotional abnormality.

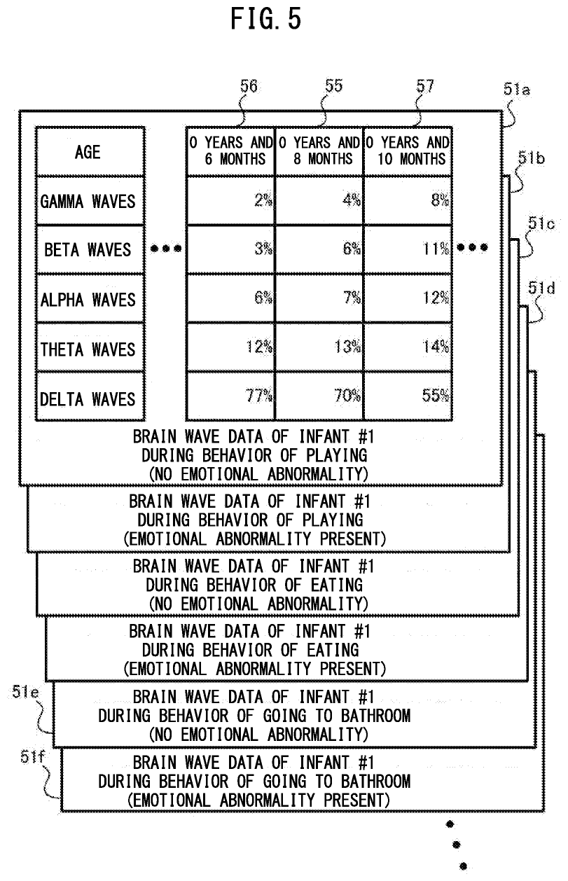

FIG. 5 is a diagram for explaining a processing example of estimating the brain wave data 51 of the specified infant 2 #1 in a recent or current period Tc, using brain wave data measured in a past period Tp1 and a past period Tp2. The past period Tp1 is a period in which the age of the infant 2 #1 is 0 years and 8 months, the past period Tp2 is a period in which the age of the infant 2 #1 is 0 years and 6 months, and the recent or current period Tc is a period in which the age of the infant 2 #1 is 0 years and 10 months. In FIG. 5, there are shown brain wave data 51a and 51b of the infant 2 #1 during a behavior in which the infant is playing, and when there is no emotional abnormality and when there is an emotional abnormality, brain wave data 51c and 51d of the infant 2 #1 during a behavior in which the infant is eating, and when there is no emotional abnormality and when there is an emotional abnormality, and brain wave data 51e and 51f of the infant 2 #1 during a behavior in which the infant is going to the bathroom, and when there is no emotional abnormality and when there is an emotional abnormality. It should be noted that the brain wave data of the infant 2 #1 may also be obtained during other behaviors, and when there is no emotional abnormality and when there is an emotional abnormality.

As illustrated in FIG. 5, in the brain wave data 51a of the infant 2 #1 at a time during a behavior in which the infant is playing and there is no emotional abnormality, a record 55 of actual measured values and a record 56 of actual measured values of the brain wave data 51 which are respectively collected by the brain wave sensor 5 from the infant 2 #1 during a behavior in which the infant is playing and when there is no emotional abnormality in the periods in which the age of the infant is 0 years and 8 months and 0 years and 6 months are obtained based on the brain wave data 51 of the infant 2 #1 illustrated in FIG. 3(b). In accordance with the record 55 of the actual measured values of the brain wave data 51, among the brain waves 50 of the infant 2 #1, the gamma waves occupy a percentage of 4%, the beta waves occupy a percentage of 6%, the alpha waves occupy a percentage of 7%, the theta waves occupy a percentage of 13%, and the delta waves occupy a percentage of 70%, respectively. In accordance with the record 56 of the actual measured values of the brain wave data 51, among the brain waves 50 of the infant 2 #1, the gamma waves occupy a percentage of 2%, the beta waves occupy a percentage of 3%, the alpha waves occupy a percentage of 6%, the theta waves occupy a percentage of 12%, and the delta waves occupy a percentage of 77%, respectively.

When a record of recent or current actual measured values included in the period in which the age of the infant 2 #1 is 0 years and 10 months does not exist within the brain wave data 51 of the infant 2 #1 during a behavior in which the infant is playing with no emotional abnormality, then using actual measured values of the brain wave data measured in a past period, and the reference information 53 illustrated in FIG. 4, it is possible to obtain a record 57 of estimated values of the brain wave data 51 in which the age of the infant 2 #1 during a behavior in which the infant is playing with no emotional abnormality is 0 years and 10 months.

In order to obtain the record 57 of the estimated values, the biological information estimation unit 213 included in the processor 210 of the childcare assistance device 20 extracts the above-described record 55 and record 56 of the actual measured values of the brain wave data 51 of the infant 2 #1, and the reference value table 53a corresponding to a behavior in which the infants 2 are playing with no emotional abnormality, which is generated based on the brain waves 50 measured during a behavior in which the plurality of infants 2 are playing and when there is no emotional abnormality. The record 55 and the record 56 of the actual measured values of the brain wave data 51 of the infant 2 #1 shown in FIG. 5, which correspond respectively to the period in which the age of the infant 2 #1 is 0 years and 8 months and the period in which the age of the infant 2 #1 is 0 years and 6 months, coincide with the reference values for the period of 0 years and 10 months and the period of 0 years and 8 months in the reference value table 53a corresponding to a behavior in which the infants 2 are playing with no emotional abnormality shown in FIG. 4. In calculating a degree of coincidence or a degree of similarity for indicating which of the reference values the past actual measured values of the brain wave data 51 of the infant 2 #1 coincide with or are similar to, for example, calculation of a least square sum based on values of the respective frequency component percentages, calculation of a Euclidean distance based on the values and the standard deviation of the respective frequency component percentages, or alternatively, an output of a learning model in which machine learning is executed in order to generate the reference information 53 using the accumulated information 52 stored in the storage device 25 as teacher data is used.

According to the examples shown in FIGS. 4 and 5, the growth level of the infant 2 #1 during a behavior of playing is two months earlier than the average age of the plurality of infants 2. Thus, using the records 55 and 56 of the actual measured values of the brain wave data 51 in the past periods in which the ages of the infant 2 #1 are 0 years and 8 months and 0 years and 6 months, and the reference value table 53a corresponding to the behavior in which the plurality of infants 2 are playing with no emotional abnormality, the biological information estimation unit 213 obtains the record 57 of the estimated values of the brain wave data 51 in which the age of the infant 2 #1 during a behavior in which the infant is playing with no emotional abnormality is 0 years and 10 months.

Moreover, the records 55 and 56 of the actual measured values of the brain wave data 51 in the past periods in which the ages of the infant 2 #1 are 0 years and 8 months and 0 years and 6 months during a behavior in which the infant is playing and when there is no emotional abnormality, as described above, are obtained based on the brain wave data 51 of the infant 2 #1 illustrated in FIG. 3(b). The brain wave data 51 of the infant 2 #1 illustrated in FIG. 3(b) is associated with the behavior information and the emotional information acquired in the past period by the additional information acquisition unit 215 included in the processor 210 of the childcare assistance device 20. Accordingly, a record in which the behavior information indicates a “behavior of playing,” and the emotional information indicates “no emotional abnormality” is selected from the brain wave data 51 in the past periods in which the ages of the infant 2 #1 is 0 years and 8 months and 0 years and 6 months, and based on the selected record, the records 55 and 56 of the actual measured values of the brain wave data 51 illustrated in FIG. 5 are obtained.

The record 57 of the estimated values of the brain wave data 51 of the infant 2 #1 having an age of 0 years and 10 months, for example, is obtained as values that coincide with or are similar to the reference values of the brain wave data for the period of 1 year and 0 months in the reference value table 53a shown in FIG. 4 corresponding to a behavior in which average infants 2 are playing with no emotional abnormality. In accordance with the record 57 of the actual measured values of the brain wave data 51 thus obtained, as shown in FIG. 5, among the brain waves 50 of the infant 2 #1, the gamma waves occupy a percentage of 8%, the beta waves occupy a percentage of 11%, the alpha waves occupy a percentage of 12%, the theta waves occupy a percentage of 14%, and the delta waves occupy a percentage of 55%, respectively. The brain wave data 51a of the infant 2 #1 during a behavior of playing and when there is no emotional abnormality is made to include the records 55 and 56 of the actual measured values in the past periods, and the record 57 of the estimated values in a recent or current period, as obtained in the manner described above.

Similarly, using the brain wave data 51 of the infant 2 #1 in a past period during a behavior of playing and when there is an emotional abnormality, and the reference value table 53b of the brain wave data of the plurality of infants 2 during a behavior of playing and when there is an emotional abnormality, the biological information estimation unit 213 obtains a record of estimated values of the brain wave data 51 of the infant 2 #1 during a behavior of playing and when there is an emotional abnormality in the period in which the age of the infant is 0 years and 10 months. Further, using the brain wave data 51 of the infant 2 #1 in a past period during a behavior of eating and when there is no emotional abnormality and when there is an emotional abnormality, and the reference value table 53c and 53d of the brain wave data of the plurality of infants 2 during a behavior of eating and when there is no emotional abnormality and when there is an emotional abnormality, the biological information estimation unit 213 obtains a record of estimated values of the brain wave data 51 of the infant 2 #1 during a behavior of eating and when there is no emotional abnormality and when there is an emotional abnormality in the period in which the age of the infant is 0 years and 10 months. Further, using the brain wave data 51 of the infant 2 #1 in a past period during a behavior of going to the bathroom and when there is no emotional abnormality and when there is an emotional abnormality, and the reference value tables 53e and 53f of the brain wave data of the plurality of infants 2 during a behavior of going to the bathroom and when there is no emotional abnormality and when there is an emotional abnormality, the biological information estimation unit 213 obtains a record of estimated values of the brain wave data 51 of the infant 2 #1 during a behavior of going to the bathroom and when there is no emotional abnormality and when there is an emotional abnormality in the period in which the age of the infant is 0 years and 10 months. Furthermore, records of estimated values of the brain wave data 51 of the infant 2 #1 may be obtained respectively during various other behaviors and when there is no emotional abnormality and when there is an emotional abnormality in the period in which the age of the infant is 0 years and 10 months. In this manner, records of the estimated values of the brain wave data 51 of the infant 2 #1 in the period in which the age of the infant is 0 years and 10 months are obtained for each of combinations of the type of behavior of the infant 2 #1 and the presence or absence of the occurrence of an emotional abnormality.

For example, it is assumed that the infant 2 #1 having an age of 0 years and 10 months as of Oct. 18, 2017 is cared for by the babysitter 3 during the period from 9:00 to 18:00 on that day. At that time, the brain wave data 51 of the infant 2 #1 during that period is measured by the infant 2 #1 wearing the brain wave sensor 5, and is acquired by the biological information acquisition unit 211 included in the processor 210 of the childcare assistance device 20. The determination unit 214 included in the processor 210 of the childcare assistance device 20 compares the acquired brain wave data 51 of the infant 2 #1, and the record of a plurality of estimated values of the brain wave data 51 of the infant 2 #1 having an age of 0 years and 10 months. Moreover, the plurality of estimated values of the brain wave data 51 of the infant 2 #1 having an age of 0 years and 10 months, as described previously, are a plurality of estimated values of the brain wave data 51 which are obtained corresponding respectively to various behaviors of the infant 2 #1 that can be expected.

As a result of such a comparison process, when the degree of coincidence or the degree of similarity between the brain wave data 51 of the infant 2 #1 acquired during a specified period within the period in which the babysitter 3 is providing childcare for the infant 2 #1, and for example, the estimated value of the brain wave data 51 of the infant 2 #1 during a behavior of eating and when there is no emotional abnormality is the highest, the determination unit 214 determines that the behavior of the infant 2 #1 during such a specified period corresponds to the behavior of eating and that there is no emotional abnormality. Further, when the degree of coincidence or the degree of similarity between the brain wave data 51 of the infant 2 #1 acquired during the specified period within the period in which the babysitter 3 is providing childcare for the infant 2 #1, and for example, the estimated value of the brain wave data 51 of the infant 2 #1 during a behavior of eating and when there is an emotional abnormality is the highest, the determination unit 214 determines that the behavior of the infant 2 #1 during such a specified period corresponds to the behavior of eating and that there is an emotional abnormality. Moreover, in calculating the degree of coincidence or the degree of similarity, for example, calculation of a least square sum based on values of the respective frequency component percentages, calculation of a Euclidean distance based on the values and the standard deviation of the respective frequency component percentages, or alternatively, an output of a learning model in which machine learning is executed in order to determine the behavior of the infants 2 using the accumulated information 52 stored in the storage device 25 as teacher data is used.

FIG. 6 is a diagram illustrating a determination result of a behavior and an emotional abnormality of the specified infant 2 #1, and a display screen 140 of the electronic device 11 that has received notification information 141 for notifying the babysitter 3 of the determination result. As of Oct. 18, 2017, the age of the infant 2 #1 is 0 years and 10 months. As noted previously, the behavior of the infant 2 #1 performed during the period in which the babysitter 3 is providing childcare for the infant 2 #1, and the presence or absence of the occurrence of an emotional abnormality of the infant 2 #1 when such a behavior is performed are determined by the determination unit 214 included in the processor 210 of the childcare assistance device 20. An example of such a determination result is shown in FIG. 6(a).

According to FIG. 6(a), in the period from 9:00 to 11:35 on Oct. 18, 2017, the behavior of the infant 2 #1 was determined to be a behavior of sleeping, and it was determined that there was no emotional abnormality of the infant 2 #1. In the period from 11:35 to 11:50 on Oct. 18, 2017, the behavior of the infant 2 #1 was determined to be a behavior of eating, and it was determined that there was an emotional abnormality of the infant 2 #1. In the period from 11:50 to 12:00 on Oct. 18, 2017, the behavior of the infant 2 #1 was determined to be a behavior of going to the bathroom, and it was determined that there was no emotional abnormality of the infant 2 #1. In the period from 12:00 to 14:00 on Oct. 18, 2017, the behavior of the infant 2 #1 was determined to be a behavior of playing, and it was determined that there was no emotional abnormality of the infant 2 #1. In the period from 14:00 to 16:10 on Oct. 18, 2017, the behavior of the infant 2 #1 was determined to be a behavior of sleeping, and it was determined that there was no emotional abnormality of the infant 2 #1. In the period from 16:10 to 16:20 on Oct. 18, 2017, the behavior of the infant 2 #1 was determined to be a behavior of going to the bathroom, and it was determined that there was no emotional abnormality of the infant 2 #1. In the period from 16:20 to 16:40 on Oct. 18, 2017, the behavior of the infant 2 #1 was determined to be a behavior of eating, and it was determined that there was an emotional abnormality of the infant 2 #1. In the period from 16:40 to 18:00 on Oct. 18, 2017, the behavior of the infant 2 #1 was determined to be a behavior of playing, and it was determined that there was no emotional abnormality of the infant 2 #1.

The determination result, shown in FIG. 6(a), of the behaviors and the emotional abnormalities of the infant 2 #1 made by the determination unit 214 can be referred to by the parent of the infant 2 #1 operating the electronic device 10 and/or by the babysitter 3 operating the electronic device 11, together with the caregiver information which indicates that the caregiver of the infant 2 #1 in the period from 9:00 to 18:00 on Oct. 18, 2017 for which the determination result is shown was the babysitter 3. The notification information generating unit 216 included in the processor 210 of the childcare assistance device 20 may create notification information for notifying the parent and/or the babysitter 3 of the infant 2 #1 of the determination result of the behaviors and the emotional abnormalities of the infant 2 #1 made by the determination unit 214, together with the caregiver information. When the notification information generating unit 216 transmits the created notification information to the electronic device 10 and/or the electronic device 11 via the communication module 230, the electronic device 10 and/or the electronic device 11 can display the received notification information on a screen. Furthermore, based on the determination result of the behaviors and the emotional abnormalities of the infant 2 #1 in the period from 9:00 to 18:00 on Oct. 18, 2017, the determination unit 214 included in the processor 210 of the childcare assistance device 20 may determine whether or not the babysitter 3 was suitable to provide childcare for the infant 2 #1 during that period, and such a childcare suitability determination result may be included in the above-described notification information created by the notification information generating unit 216.

According to the example shown in FIG. 6(a), in the period from 11:35 to 11:50 and in the period from 16:20 to 16:40 on Oct. 18, 2017, the determination result of the behavior of the infant 2 #1 by the determination unit 214 in both cases is a behavior of eating, and the determination result of the emotional abnormality of the infant 2 #1 in both cases is that there is an abnormality. It may be considered that, when the babysitter 3 provides milk as food to the infant 2 #1, the baby bottle that is held in the mouth of the infant 2 #1 is not tilted properly, whereby the infant 2 #1 is placed under stress.

Thus, the notification information 141, which notifies the babysitter 3 or a manager of the babysitter 3 that an emotional abnormality of the infant 2 #1 has been determined by the determination unit 214 included in the processor 210 of the childcare assistance device 20, may be generated by the notification information generating unit 216 included in the processor 210 of the childcare assistance device 20. The generated notification information 141 is transmitted by way of signal communication to the electronic device 11. The notification information 141 is displayed on the screen 140 of the electronic device 11 which has received the notification information 141. In the example shown in FIG. 6(b), as the notification information 141, a message stating that “The child seems to be feeling stress when drinking milk. Please make an effort to improve this situation by paying attention to the way the milk bottle is tilted” is displayed. More specifically, the notification information 141 may include not only a determination of an emotional abnormality of the infant 2 #1 by the determination unit 214, but also a proposal for a countermeasure to overcome the emotional abnormality of the infant 2 #1.

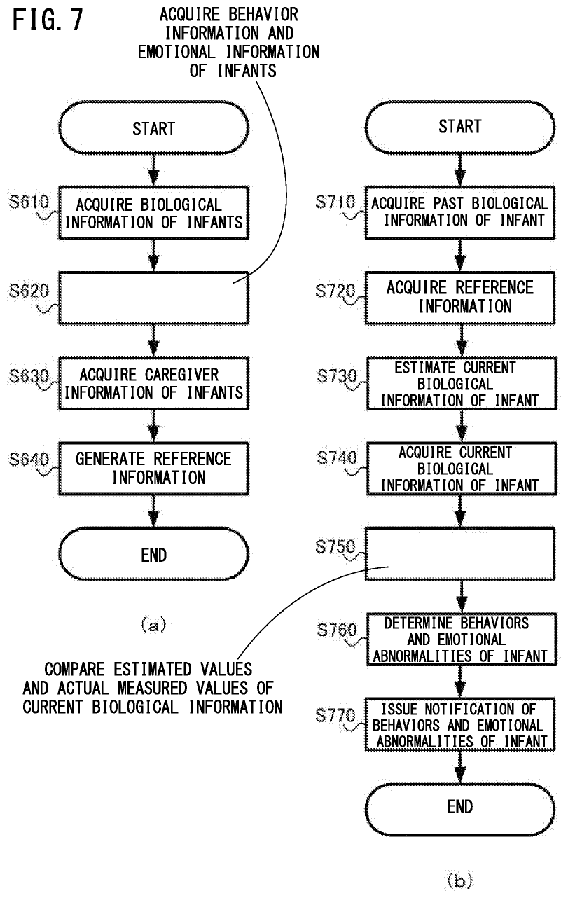

FIG. 7 is a diagram for describing an example of a reference information generation process and a behavior and emotional abnormality determination process for the infants 2 executed by running a computer program in the childcare assistance device 20 according to the present embodiment. The processor 210 of the childcare assistance device 20, by activating a computer program stored in the memory 220, and by executing the reference information generation process shown in FIG. 7(a), functions as the biological information acquisition unit 211, the reference information generating unit 212, and the additional information acquisition unit 215. In the present embodiment, such a reference information generation process is repeatedly executed.

When the reference information generation process shown in FIG. 7(a) is initiated, then in step S610, the biological information acquisition unit 211 acquires the brain wave data 51 as biological information of each of the plurality of infants 2, stores the brain wave data 51 in the storage device 25, and stores at least a portion of the acquired brain wave data 51 as the accumulated information 52.

When the behavior information indicating the behaviors of the infants 2 in the past period Tp1 and the past period Tp2 in which the brain wave data 51 of each of the infants 2 was measured, and the emotional information indicating whether or not an emotional abnormality has occurred in the infants 2 during the behaviors are input to the electronic device 10 possessed by the parents of the respective infants 2, and transmitted to the childcare assistance device 20, then in step S620, the additional information acquisition unit 215 acquires the behavior information and the emotional information of each of the infants 2 via the communication module 230. The additional information acquisition unit 215 stores the acquired behavior information and emotional information of each of the infants 2 in the storage device 25 in association with the brain wave data 51 acquired in step S610.

When the caregiver information indicating the caregiver of the infants 2 in the past period Tp1 and the past period Tp2 in which the brain wave data 51 of each of the infants 2 was measured is input to the electronic device 10 possessed by the parents of the respective infants 2, and transmitted to the childcare assistance device 20, then in step S630, the additional information acquisition unit 215 acquires the caregiver information of each of the infants 2 via the communication module 230. The additional information acquisition unit 215 stores the acquired caregiver information of each of the infants 2 in the storage device 25 in association with the brain wave data 51 acquired in step S610.

In step S640, from among the brain wave data of the plurality of infants 2 included in the accumulated information 52 stored in the storage device 25, based on the brain wave data 51 of the respective infants 2 in the period in which the caregivers of the respective infants 2 are the parents of the infants 2, the reference information generating unit 212 generates, as the reference information 53, the plurality of reference value tables 53a, 53b, 53c, etc., in accordance with the types of behavior information, and stores the reference value tables in the storage device 25. When the process of step S640 is completed, the reference information generation process shown in FIG. 7(a) comes to an end. The processes in the steps S610, S620, S630, and S640 included in the reference information generation process shown in FIG. 7(a) may operate as independent processes, respectively. For example, the process in step S610 is repeatedly executed every minute, and the processes in steps S620 and S630 are repeatedly executed each time that the behavior information, the emotional information, and the caregiver information of the infants 2 are transmitted from the electronic device 10 to the childcare assistance device 20, and the process in step S640 is repeatedly executed every week.

The processor 210 of the childcare assistance device 20, by activating a computer program stored in the memory 220, and by executing the behavior and emotional abnormality determination process for the infants 2 shown in FIG. 7(b), functions as the biological information acquisition unit 211, the biological information estimation unit 213, the determination unit 214, the additional information acquisition unit 215, and the notification information generating unit 216. In the present embodiment, the behavior and emotional abnormality determination process for the infants 2 is repeatedly executed.

When the behavior and emotional abnormality determination process for the infants 2 shown in FIG. 7(b) is initiated, then in step S710, from among the brain wave data 51 of the plurality infants 2 acquired by the biological information acquisition unit 211 and stored in the storage device 25, the biological information estimation unit 213 acquires, as respective biological information of the specified infant 2 #1, the brain wave data 51 of the infant 2 #1 in the past periods Tp1 and Tp2.

In step S720, the biological information estimation unit 213 acquires, from the storage device 25, the reference information 53 generated through the reference information generation process shown in FIG. 7(a).

In step S730, using the records 55 and 56 of the actual measured values of the brain wave data 51 of the infant 2 #1 in the past periods Tp1 and Tp2 acquired in step S710, and the reference information 53 acquired in step S720, the biological information estimation unit 213 estimates the brain wave data 51 of the infant 2 #1 in the recent or current period Tc, and stores the record 57 of the estimated values in the storage device 25.

In step S740, the biological information acquisition unit 211 acquires a record of the actual measured values of the brain wave data 51 of the infant 2 #1 in the recent or current period Tc, and stores the record in the storage device 25. In addition, the additional information acquisition unit 215 may acquire the behavior information, the emotional information, and the caregiver information of each of the infants 2 via the communication module 230, and may store the acquired behavior information, emotional information, and caregiver information of each of the infants 2 in the storage device 25 in association with the record of the actual measured values thereof.

In step S750, the determination unit 214 compares the record of the actual measured values acquired in step S740, and the record 57 of the estimated values acquired in step S730, of the brain wave data 51 of the infant 2 #1 in the recent or current period Tc.

In step S760, the determination unit 214 determines the behaviors and the emotional abnormalities of the infant 2 #1 in the recent or current period Tc, based on the comparison result, acquired in step S750, between the record of the actual measured values and the record 57 of the estimated values, of the brain wave data 51 of the infant 2 #1.

In step S770, the notification information generating unit 216 generates notification information for notifying the parent of the infant 2 #1 and the babysitter 3 of the determination result of the behaviors and the emotional abnormalities of the infant 2 #1 obtained in step S760. The notification information generating unit 216 transmits the generated notification information via the communication module 230 to the electronic device 10 that is possessed by the parent of the infant 2 #1 and the electronic device 11 that is possessed by the babysitter 3 or a manager of the babysitter 3. At that time, if the behavior information, the emotional information, and the caregiver information of each of the infants 2 are stored in the storage device 25 in step S740, the notification information is transmitted together with the behavior information, the emotional information, and the caregiver information. As noted previously, the childcare suitability determination result of the babysitter 3 with respect to the infant 2 #1 as determined by the determination unit 214 and/or information concerning a proposal for a countermeasure to overcome the emotional abnormality of the infant 2 #1 may be included in the notification information. When the process of step S770 is completed, the behavior and emotional abnormality determination process for the infants 2 shown in FIG. 7(b) comes to an end.

According to the childcare assistance device 20 in the present embodiment, the following advantageous effects can be obtained.

(1) By activating the computer program stored in the memory 220, the processor 210 of the childcare assistance device 20 logically includes at least the biological information acquisition unit 211, the biological information estimation unit 213, and the determination unit 214. The biological information acquisition unit 211 acquires the brain wave data 51 as biological information of the infant 2 #1. Using the brain wave data 51 of the infant 2 #1 in the past period Tp1 acquired by the biological information acquisition unit 211, and the reference information 53 based on the brain wave data of the plurality of infants 2 #X1, #X2, □, #Xn, the biological information estimation unit 213 estimates the brain wave data 51 of the infant 2 #1 in the recent or current period Tc that differs from the past period Tp1. The determination unit 214 determines an emotional abnormality of the infant 2 #1 in the recent or current period Tc, on the basis of the comparison between the actual measured values of the brain wave data 51 of the infant 2 #1 in the recent or current period Tc acquired by the biological information acquisition unit 211, and the estimated values of the brain wave data 51 of the infant 2 #1 estimated by the biological information estimation unit 213. Therefore, according to the childcare assistance device 20, it is highly possible that emotional abnormalities of the various infants 2 of different ages can be appropriately determined for each of the individual infants 2.

(2) In the childcare assistance device 20, the biological information acquisition unit 211 acquires brain wave data of the plurality of infants 2 #X1, #X2, □, #Xn. The reference information 53 is obtained based on the accumulated information 52 in which the brain wave data of the plurality of infants 2 #X1, #X2, □, #Xn acquired by the biological information acquisition unit 211 is included. Therefore, according to the childcare assistance device 20, there is a high possibility that emotional abnormalities of the infant 2 #1 can be accurately determined.

(3) In the childcare assistance device 20, using the actual measured values of the brain wave data 51 of the infant 2 #1 in the past period Tp1 acquired by the biological information acquisition unit 211, the actual measured values of the brain wave data 51 of the infant 2 #1 in the past time period Tp2 that differs from both the period Tp1 and the period Tc, which is acquired by the biological information acquisition unit 211, and the reference information 53, the biological information estimation unit 213 estimates the brain wave data 51 of the infant 2 #1 in the recent or current period Tc. Therefore, according to the childcare assistance device 20, there is a high possibility that emotional abnormalities of the infant 2 #1 can be more appropriately determined taking into consideration a growth level peculiar to the infant 2 #1.

(4) The processor 210 of the childcare assistance device 20 further logically includes the additional information acquisition unit 215. The additional information acquisition unit 215 acquires the behavior information indicating the behavior of the infant 2 #1 in the past period Tp1 in which the brain wave data 51 of the infant 2 #1 was measured, and associates the behavior information with the brain wave data 51 of the infant 2 #1 in the past period Tp1 acquired by the biological information acquisition unit 211. Using the brain wave data 51 of the infant 2 #1 in the past period Tp1 with which the behavior information is associated by the additional information acquisition unit 215, and the reference information 53 that is based on the brain wave data 51 of the infant 2 #1 in the past period Tp1 and corresponds to the type of behavior information, the biological information estimation unit 213 estimates the brain wave data 51 of the infant 2 #1 in the recent or current period Tc for each of the types of behavior information. The determination unit 214 determines the emotional abnormality of the infant 2 #1 in the period Tc and the behavior of the infant 2 #1 in the period Tc, on the basis of the comparison between the actual measured values of the brain wave data 51 of the infant 2 #1 in the recent or current period Tc acquired by the biological information acquisition unit 211, and the estimated values of the brain wave data 51 of the infant 2 #1 in the recent or current period Tc estimated for each of the types of behavior information by the biological information estimation unit 213. Therefore, according to the childcare assistance device 20, there is a high possibility that a state of the childcare can be confirmed after completion of a temporary childcare, without monitoring the childcare for the infant 2 #1 provided by the babysitter 3.

(5) The additional information acquisition unit 215 included in the processor 210 of the childcare assistance device 20 acquires the emotional information indicating whether or not the emotional abnormality has occurred in the infant 2 #1 during the behavior, for example, of eating or the like in the past period Tp1 in which the brain wave data 51 of the infant 2 #1 was measured, and associates the acquired emotional information with the brain wave data 51 of the infant 2 #1 in the past period Tp1 acquired by the biological information acquisition unit 211. Using the brain wave data 51 of the infant 2 #1 in the past period Tp1 with which the emotional information is associated by the additional information acquisition unit 215, and the reference information 53 that is based on the brain wave data of the plurality of infants 2 #X1, #X2, □, #Xn and corresponds to the type of behavior information of the infants 2 and the presence or absence of the occurrence of an emotional abnormality, the biological information estimation unit 213 estimates the brain wave data 51 of the infant 2 #1 in the recent or current period Tc for each of combinations of the type of behavior information and the presence or absence of the occurrence of an emotional abnormality. The determination unit 214 determines the behaviors and the emotional abnormalities of the infant 2 #1 in the period Tc, based on the comparison between the brain wave data 51 of the infant 2 #1 in the period Tc acquired by the biological information acquisition unit 211, and the brain wave data 51 of the infant 2 #1 in the period Tc, which is estimated for each of combinations of the type of behavior information and the presence or absence of the occurrence of an emotional abnormality by the biological information estimation unit 213. Therefore, according to the childcare assistance device 20, there is a high possibility that emotional abnormalities of the infant 2 #1 can be highly accurately determined.

(6) The processor 210 of the childcare assistance device 20 further includes the notification information generating unit 216. The notification information generating unit 216 generates the notification information for notifying the babysitter 3 who is the caregiver of the infant 2 #1 in the period Tc or a manager of the babysitter 3 via the electronic device 11 that an emotional abnormality of the infant 2 #1 has been determined by the determination unit 214. Therefore, according to the childcare assistance device 20, there is a high possibility that the babysitter 3 will be capable of obtaining information necessary for improving a skill at providing childcare and enhancing the evaluation.

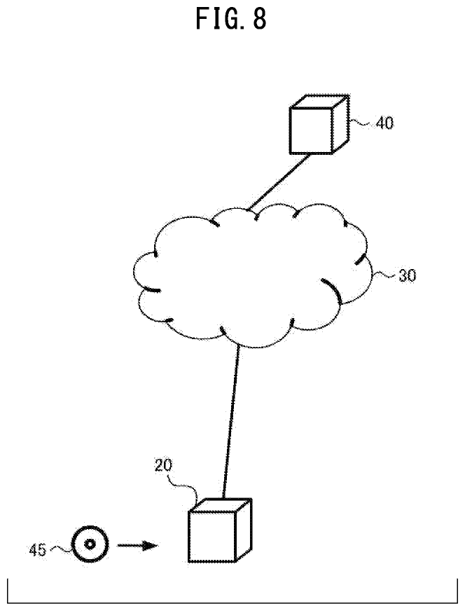

FIG. 8 is a diagram illustrating a product supplying mode of a computer program that runs in the childcare assistance device 20 according to the above-described embodiment. The computer program that runs in the childcare assistance device 20 can be supplied to the childcare assistance device 20 through a recording medium 45 such as a CD-ROM or a USB flash drive, or through a data signal that flows over the communication network 30 such as the Internet.

A computer program providing server 40 is a server computer that supplies the aforementioned computer program, and stores the computer program in a storage device such as a hard disk or the like. The communication network 30 is the Internet, a wireless LAN, a telephone network, or alternatively, a dedicated line or the like. The computer program providing server 40 reads out the computer program that is stored in the storage device, places it in the form of a data signal on a carrier wave, and transmits the data signal to the childcare assistance device 20 via the communication network 30. In this manner, the computer program can be supplied as a computer-readable computer program product in various formats such as a recording medium, a data signal, or the like.

The following modifications also lie within the scope of the present invention, and one or plurality of exemplary modifications can also be combined with the above-described embodiment.

(Exemplary Modification 1) Although in the above-described embodiment, all of the plurality of reference value tables that make up the reference information 53 generated by the reference information generating unit 212 included in the processor 210 of the childcare assistance device 20 are generated for each of combinations of the type of behavior information of the infants 2 and the presence or absence of an emotional abnormality, the reference value tables may be generated for each of the types of the behavior information of the infants 2, regardless of the presence or absence of an emotional abnormality. When the brain wave data acquired when emotional abnormalities of the infants 2 occur is small, the reference value tables generated for each of the types of behavior information of the infants 2 are considered to correspond substantially to reference value tables corresponding to the respective types of behaviors of the infants 2 with no emotional abnormality. In the present Exemplary Modification 1, using the reference information 53 constituted by the plurality of reference value tables thus obtained based on the brain wave data of the plurality of infants 2, and the brain wave data 51, in the past period Tp1, of the infant 2 #1 who is an estimation target infant 2 of the brain wave data 51, the biological information estimation unit 213 included in the processor 210 of the childcare assistance device 20 estimates the brain wave data 51 of the infant 2 #1 in the recent or current period Tc that differs from the past period Tp1. The record of the estimated values of the brain wave data 51 of the infant 2 #1 in the period Tc, which is estimated in this manner, is considered to correspond substantially to the brain wave data 51 of the infant 2 #1 during the respective types of behaviors of the infant 2 #1 and when there is no emotional abnormality.

The determination unit 214 included in the processor 210 of the childcare assistance device 20 compares the record of the actual measured values of the brain wave data 51 of the infant 2 #1, which is acquired by the biological information acquisition unit 211 included in the processor 210 of the childcare assistance device 20, during a period within the period Tc in which the babysitter 3 is providing childcare for the infant 2 #1, and the record of the estimated values of the brain wave data 51 of the infant 2 #1 in the period Tc, which is estimated by the biological information estimation unit 213. As a result of such a comparison, when the degree of coincidence or the degree of similarity between the actual measured values of the brain wave data 51 of the infant 2 #1 acquired by the biological information acquisition unit 211, and the estimated values of the brain wave data 51 of the infant 2 #1, for example, during a behavior of eating, is highest in comparison with the degree of coincidence or the degree of similarity with the estimated values of the brain wave data 51 of the infant 2 #1 during other types of behavior, the determination unit 214 determines that the behavior of the infant 2 #1 during the period in which the babysitter 3 is providing childcare for the infant 2 #1 corresponds to the behavior of eating. Further, when the degree of coincidence or the degree of similarity is lower than a predetermined value, it is considered that an emotional abnormality such as stress has occurred when the infant 2 #1 is engaged in the behavior of eating, and therefore, the determination unit 214 determines that there is an emotional abnormality of the infant 2 #1 in such a specified period. When the degree of coincidence or the degree of similarity is higher than the predetermined value, it is considered that no emotional abnormality such as stress has occurred when the infant 2 #1 is engaged in the behavior of eating, and therefore, the determination unit 214 determines that there is no emotional abnormality of the infant 2 #1 in such a specified period. Calculation of the degree of coincidence or the degree of similarity is performed in the same manner as the calculation in the above-described embodiment.

(Exemplary Modification 2) In the above-described embodiment, the behavior information of the infants 2 acquired by the additional information acquisition unit 215 included in the processor 210 of the childcare assistance device 20 is input to the electronic device 10 and transmitted from the electronic device 10 to the childcare assistance device 20. In the Exemplary Modification 2, an image capturing device such as a camera is arranged which captures images of the infants 2 as photographic subjects. The images of the infants 2 as the photographic subjects are extracted from the images obtained by the image capturing device, and the behaviors of the infants 2 can be specified through an image recognition process such as pattern matching, or image classification according to a machine-learned learning model. The additional information acquisition unit 215 acquires the behavior information indicating the behaviors of the infants 2 identified in this manner, together with time information indicating the time when the images were captured, and associates the acquired behavior information of the infants 2 with the brain waive data of the infants 2 stored in the storage device 25 and corresponding to such time information.

(Exemplary Modification 3) In the above-described embodiment, the plurality of functional units included in the processor 210 of the childcare assistance device 20 are illustrated in FIG. 2, but a portion or all of the plurality of functional units included in the processor 210 may be included in the electronic device 10 and/or the electronic device 11. In the Exemplary Modification 3, among the plurality of functional units included in the processor 210, the biological information estimation unit 213, the determination unit 214, and the notification information generating unit 216 are included in the electronic device 10 and/or the electronic device 11. In this case, the reference information 53 generated by the reference information generating process shown in FIG. 7(a), which is executed in the childcare assistance device 20, is stored in the electronic device 10 and/or the electronic device 11 via the communication network 30, and using the reference information 53, the behavior and emotional abnormality determination process of the infants 2 shown in FIG. 7(b) is executed in the electronic device 10 and/or the electronic device 11.

(Exemplary Modification 4) In the above-described embodiment, the determination unit 214 included in the processor 210 of the childcare assistance device 20 may determine the childcare suitability determination result of the babysitter 3 with respect to the infant 2 #1. The notification information generating unit 216 included in the processor 210 of the childcare assistance device 20 generates notification information including the childcare suitability determination result of the babysitter 3 determined by the determination unit 214, and transmits the created notification information to the electronic device 10 possessed by the parent of the infant 2 #1 via the communication module 230. The determination criteria for the childcare suitability determination of the babysitter 3 with respect to the infant 2 #1 can be arbitrarily determined, however, according to the present Modification 4, a relative childcare suitability determination is carried out by performing a babysitter evaluation process based on the brain wave data of the infant 2 #1 when childcare is provided by a plurality of babysitters.

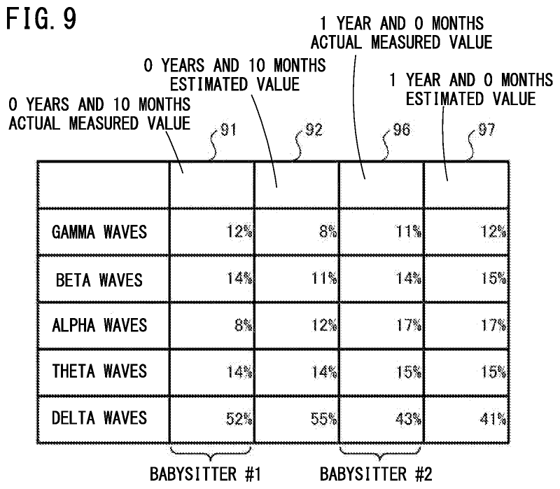

FIG. 9 is a diagram for describing an example of a babysitter evaluation process executed by the childcare assistance device 20 in the Exemplary Modification 4. In FIG. 9, four types of records 91, 92, 96, and 97 are illustrated. The record 91 is a record of actual measured values of the brain wave data 51 of the infant 2 #1, which is measured by the brain wave sensor 5 when a babysitter 3 #1 is providing childcare for the infant 2 #1 in a period in which the age of the infant 2 #1 is 0 years and 10 months, and is acquired by the biological information acquisition unit 211 included in the processor 210 of the childcare assistance device 20. The fact that the caregiver corresponding to this record 91 is the babysitter 3 #1 is identified from the caregiver information of the infant 2 #1, which is acquired by the additional information acquisition unit 215 included in the processor 210 of the childcare assistance device 20, being associated with the record of the actual measured values 91. In the record 91, the respective frequency component percentages of the gamma waves, the beta waves, the alpha waves, the theta waves, and the delta waves that make up the brain waves 50 of the infant 2 #1 are 12%, 14%, 8%, 14%, and 52%, respectively. The record 92 is a record of estimated values of the brain wave data 51 of the infant 2 #1 in the period of 0 years and 10 months, which is estimated by the biological information estimation unit 213 included in the processor 210 of the childcare assistance device 20 using the aforementioned reference information 53. In the record 92, the respective frequency component percentages of the gamma waves, the beta waves, the alpha waves, the theta waves, and the delta waves that make up the brain waves 50 of the infant 2 #1 are 8%, 11%, 12%, 14%, and 55%, respectively.

The record 96 is a record of actual measured values of the brain wave data 51 of the infant 2 #1, which is measured by the brain wave sensor 5 when a babysitter 3 #2 is providing childcare for the infant 2 #1 in the period Tc in which the age of the infant 2 #1 is 1 year and 0 months, and is acquired by the biological information acquisition unit 211. The fact that the caregiver corresponding to this record 96 is the babysitter 3 #2 is identified from the caregiver information of the infant 2 #1, which is acquired by the additional information acquisition unit 215, being associated with the record of the actual measured values 91. In the record 96, the respective frequency component percentages of the gamma waves, the beta waves, the alpha waves, the theta waves, and the delta waves that make up the brain waves 50 of the infant 2 #1 are 11%, 14%, 17%, 15%, and 43%, respectively. The record 97 is a record of estimated values of the brain wave data 51 of the infant 2 #1 in the period of 1 year and 0 months, which is estimated by the biological information estimation unit 213 using the aforementioned reference information 53. In the record 97, the respective frequency component percentages of the gamma waves, the beta waves, the alpha waves, the theta waves, and the delta waves that make up the brain waves 50 of the infant 2 #1 are 12%, 15%, 17%, 15%, and 41%, respectively.

The determination unit 214 calculates a square sum D1 of the record 91 of the actual measured values and the record 92 of the estimated values of the brain wave data 51 of the infant 2 #1 in the period in which the age of the infant who was cared for by the babysitter 3 #1 is 0 years and 10 months, and a square sum D2 of the record 96 of the actual measured values and the record 97 of the estimated values of the brain wave data 51 of the infant 2 #1 in the period Tc in which the age of the infant who was cared for by the babysitter 3 #2 is 1 year and 0 months. It implies that, among the square sums D1 and D2, the relatively smaller one of them has a higher degree of similarity between the measured values and the estimated values. In the present Exemplary Modification 4, in order to obtain the degree of similarity between the measured values and the estimated values, the square sums D1 and D2 of the differences between the values of the respective frequency component percentages are used, however, the degree of similarity may be determined using a calculation according to a different method.

Since the square sum D1 is the square sum of the differences between the values of the frequency component percentages recorded in the records 91 and 92, the square sum D1 is calculated by the following equation (1), and the value of D1=50 is obtained. Since the square sum D2 is the square sum of the differences between the values of the frequency component percentages recorded in the records 96 and 97, the square sum D2 is calculated by the following equation (2), and the value of D2=6 is obtained.

D1=(12−8)²+(14−11)²+(8−12)²+(14−14)²+(52−55)²=50 □(1)

D2=(11−12)²+(14−15)²+(17−17)²+(15−15)²+(43−41)²=6 □(2)

According to the above-described equations (1) and (2), since the square sum D2 is smaller than the square sum D1, the square sum D2 is the least square sum, and more specifically, the degree of similarity between the actual measured values and the estimated values of the brain wave data 51 of the infant 2 #1 who was cared for by the babysitter 3 #2 is higher than the degree of similarity between the actual measured values and the estimated values of the brain wave data 51 of the infant 2 #1 who was cared for by the babysitter 3 #1. Comparing the childcare provided by the babysitter 3 #1 and the childcare provided by the babysitter 3 #2 for the infant 2 #1, it is considered that the childcare provided by the babysitter 3 #2 is less likely to cause an emotional abnormality of the infant 2 #1. Thus, as an evaluation process of the babysitters 3 with respect to the infant 2 #1, the determination unit 214 gives a relatively higher evaluation to the babysitter 3 #2 than to the babysitter 3 #1. Stated otherwise, the determination unit 214 determines that the babysitter 3 #2 is suitable to provide childcare for the infant 2 #1.

According to the childcare assistance device 20 in the present Exemplary Modification 4, the following advantageous effects can be obtained.

The caregiver information acquisition unit included in the processor 210 of the childcare assistance device 20 acquires the caregiver information indicating the caregiver of the infant 2 #1 when the brain wave data 51 of the infant 2 #1 acquired by the biological information acquisition unit 211 is measured. Based on the result of having determined the emotional abnormalities of the infant 2 #1 during the period Tc, the determination unit 214 determines whether the babysitter 3 #2, who is the caregiver of the infant 2 #1, is suitable to provide childcare for the infant 2 #1. Therefore, in accordance with the childcare assistance device 20, when the parent of the infant 2 #1 selects a babysitter 3, there is a high possibility that the babysitter 3 #2 who is relatively suitable to provide childcare for the infant 2 #1 can be selected.

(Exemplary Modification 5) In the above-described embodiment and the exemplary modifications, the determination unit 214 included in the processor 210 of the childcare assistance device 20 is capable of determining whether or not the babysitter 3 was suitable to provide childcare for the infant 2 #1. In the Exemplary Modification 5, the childcare suitability determination result of the babysitter 3 with respect to the infant 2 #1 is used for determining the childcare suitability of the babysitter 3 with respect to another infant 2 #3.

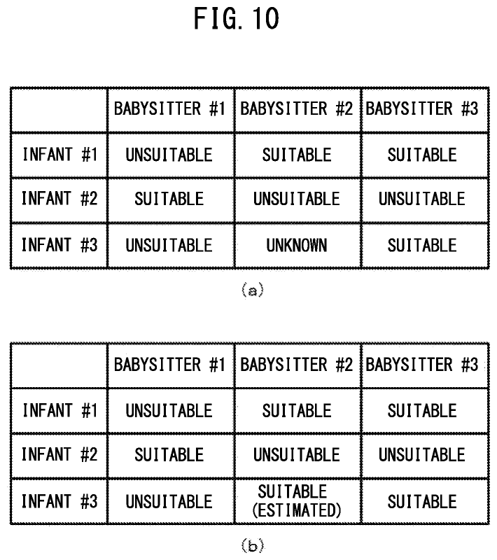

FIG. 10 is a diagram for describing an example of a babysitter evaluation process executed by the childcare assistance device 20 in the present Exemplary Modification 5. In FIG. 10(a), childcare suitability determination results of the babysitter 3 #1, the babysitter 3 #2, and a babysitter 3 #3 with respect to the infant 2 #1, an infant 2 #2, and the infant 2 #3 are shown, respectively. According to FIG. 10(a), the childcare suitability determination results of the babysitter 3 #1, the babysitter 3 #2, and the babysitter 3 #3 with respect to the infant 2 #1 are unsuitable, suitable, and suitable, respectively. The childcare suitability determination results of the babysitter 3 #1, the babysitter 3 #2, and the babysitter 3 #3 with respect to the infant 2 #2 are suitable, unsuitable, and unsuitable, respectively. The childcare suitability determination results of the babysitter 3 #1 and the babysitter 3 #3 with respect to the infant 2 #3 are unsuitable and suitable, respectively. Since the babysitter 3 #2 has never provided childcare for the infant 2 #3, the childcare suitability determination result of the babysitter 3 #2 with respect to the infant 2 #3 is unknown.

In the case it is possible for the parent of the infant 2 #3 to select the babysitter 3 #2 as a babysitter 3 who is capable of being used in the time block in which the parent desires childcare for the infant 2 #3, it is desirable if the childcare suitability of the babysitter 3 #2 with respect to the infant 2 #1 can be evaluated. In the present Exemplary Modification 5, the determination unit 214 included in the processor 210 of the childcare assistance device 20 is made capable of evaluating the babysitter 3 #2 by estimating the childcare suitability of the babysitter 3 #2 with respect to the infant 2 #3, based on the childcare suitability determination results shown in FIG. 10(a) and using collaborative filtering.

With reference to the childcare suitability determination results shown in FIG. 10(a), the childcare suitability of the babysitter 3 #1, who has a history of providing childcare for the infant 2 #3, is unsuitable for the infant 2 #1, is suitable for the infant 2 #2, and is unsuitable for the infant 2 #3. More specifically, regarding the childcare suitability determination results of the babysitter 3 #1, the result for the infant 2 #1 coincides with the result for the infant 2 #3. The childcare suitability of the babysitter 3 #3, who has a history of providing childcare for the infant 2 #3, is suitable for the infant 2 #1, is unsuitable for the infant 2 #2, and is suitable for the infant 2 #3. More specifically, regarding the childcare suitability determination results of the babysitter 3 #3 as well, similar to the childcare suitability determination results of the babysitter 3 #1, the result for the infant 2 #1 coincides with the result for the infant 2 #3.

Based on the fact that the childcare suitability determination results of the babysitter 3 #1 and the babysitter 3 #3 with respect to the infant 2 #1 coincide with the childcare suitability determination results of the babysitter 3 #1 and the babysitter 3 #3 with respect to the infant 2 #3, and the fact that the childcare suitability determination result of the babysitter 3 #2 with respect to the infant 2 #1 is suitable, the determination unit 214 determines that the childcare suitability of the babysitter 3 #2 with respect to the infant 2 #3 coincides with the childcare suitability determination result of the babysitter 3 #2 with respect to the infant 2 #1, and more specifically, estimates the babysitter 3 #2 to be suitable for the infant 2 #3. In FIG. 10(b), the childcare suitability of the babysitter 3 #2 with respect to the infant 2 #3, which is undetermined in the child suitability determination results shown in FIG. 10(a), is rewritten based on the aforementioned estimation result of being suitable.

According to the childcare assistance device 20 in the present Exemplary Modification 5, the following advantageous effects can be obtained.

Based on the determination result of whether or not the babysitter 3 #2 who is the caregiver of the infant 2 #1 in the period Tc in the second period is suitable to provide childcare for the infant 2 #1, the determination unit 214 included in the processor 210 of the childcare assistance device 20 determines whether or not the babysitter 3 #2 is suitable to provide childcare for another infant 2 #3 that differs from the infant 2 #1. Therefore, according to the childcare assistance device 20, when the parent of the infant 2 #3 selects a babysitter 3, it is possible to select the babysitter 3 #2 for whom the possibility of being suitable for the infant 2 #3 is high.

(Exemplary Modification 6) In the above-described embodiment and the exemplary modifications, the determination unit 214 included in the processor 210 of the childcare assistance device 20 determines the behavior of the infant 2 #1 in the period Tc. The behavior of the infant 2 #1 and the fact that the caregiver is the babysitter 3 in the period Tc are identified from the caregiver information and the behavior information of the infant 2, which are acquired by the additional information acquisition unit 215 included in the processor 210 of the childcare assistance device 20, being associated with the record of the actual measured values of the brain wave data 51 of the infant 2 #1. In the present Exemplary Modification 6, the childcare record of the infant 2 #1 in the period Tc created by the babysitter 3 can be inspected by comparing (cross-checking) it with the determination result by the determination unit 214 concerning the behavior of the infant 2 #1 in the period Tc.

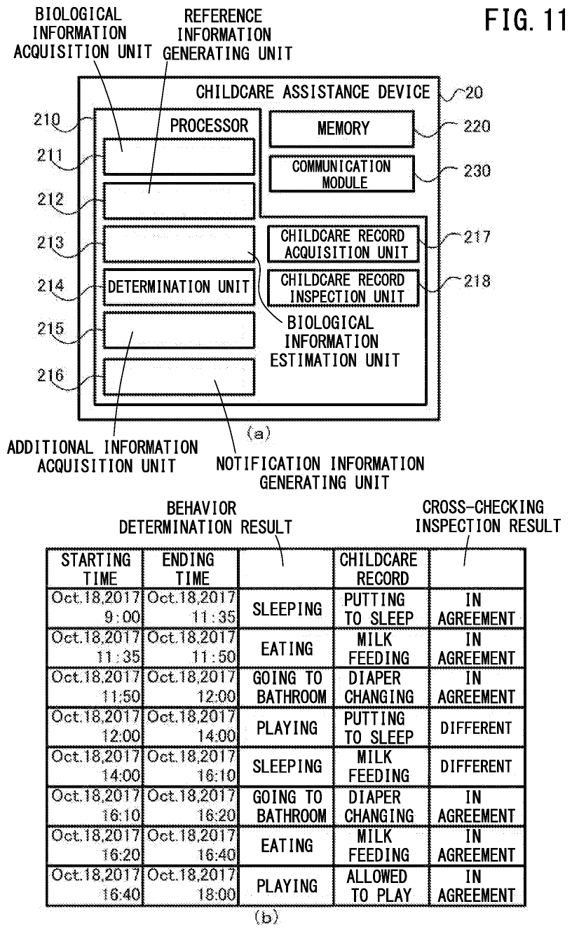

FIG. 11 is a diagram for describing an example of a babysitter childcare record inspection process executed by the childcare assistance device 20 in the present Exemplary Modification 6. The configuration of the childcare assistance device 20 in the present Exemplary Modification 6 shown in FIG. 11 (a) differs from the configuration of the childcare assistance device 20 in the aforementioned embodiment shown in FIG. 2, in that the processor 210 further includes a childcare record acquisition unit 217 and a childcare record inspection unit 218. When the babysitter 3 creates and transmits the childcare record of the infant 2 #1 in the period Tc using the electronic device 11, the childcare record acquisition unit 217 acquires the childcare record via the communication module 230. The childcare record inspection unit 218 inspects the childcare record in the period Tc acquired by the childcare record acquisition unit 217 by comparing it with the determination result of the behavior of the infant 2 #1 in the period Tc by the determination unit 214 shown in FIG. 6(a), and if there are any different items therein, such items are identified.

In FIG. 11(b), the determination result of the behavior of the infant 2 #1 in the period Tc by the determination unit 214 shown in FIG. 6(a), and the childcare record in the period Tc acquired by the childcare record acquisition unit 217 are shown. According to FIG. 11(b), in the period from 9:00 to 11:35 on Oct. 18, 2017, the behavior of the infant 2 #1 was determined to be a behavior of sleeping, and the item of “putting to sleep” is recorded in the childcare record. In the period from 11:35 to 11:50 on Oct. 18, 2017, the behavior of the infant 2 #1 was determined to be a behavior of eating, and the item of “milk feeding” is recorded in the childcare record. In the period from 11:50 to 12:00 on Oct. 18, 2017, the behavior of the infant 2 #1 was determined to be a behavior of going to the bathroom, and the item of “diaper changing” is recorded in the childcare record. Concerning the three items up to this point, as a result of the above-described cross-checking inspection, it is determined by the childcare record inspection unit 218 that the determination result of the behavior of the infant 2 #1 by the determination unit 214 and the childcare record are in agreement with each other. The cross-checking inspection result shown in FIG. 11(b) is described as “in agreement”.

In the period from 12:00 to 14:00 on Oct. 18, 2017, the behavior of the infant 2 #1 was determined to be a behavior of playing, and the item of “putting to sleep” is recorded in the childcare record. In the period from 14:00 to 16:10 on Oct. 18, 2017, the behavior of the infant 2 #1 was determined to be a behavior of sleeping, and the item of “milk feeding” is recorded in the childcare record. Concerning the two items up to this point, as a result of the above-described cross-checking inspection, it is determined by the childcare record inspection unit 218 that the determination result of the behavior of the infant 2 #1 by the determination unit 214 and the childcare record differ from each other. The cross-checking inspection result shown in FIG. 11(b) is described as “different”.

In the period from 16:10 to 16:20 on Oct. 18, 2017, the behavior of the infant 2 #1 was determined to be a behavior of going to the bathroom, and the item of “diaper changing” is recorded in the childcare record. In the period from 16:20 to 16:40 on Oct. 18, 2017, the behavior of the infant 2 #1 was determined to be a behavior of eating, and the item of “milk feeding” is recorded in the childcare record. In the period from 16:40 to 18:00 on Oct. 18, 2017, the behavior of the infant 2 #1 was determined to be a behavior of playing, and the item of “allowed to play” is recorded in the childcare record. Concerning the three items up to this point, as a result of the above-described cross-checking inspection, it is determined by the childcare record inspection unit 218 that the determination result of the behavior of the infant 2 #1 by the determination unit 214 and the childcare record are in agreement with each other. The cross-checking inspection result shown in FIG. 11(b) is described as “in agreement”.

According to the childcare assistance device 20 in the present Exemplary Modification 6, the following advantageous effects can be obtained.

The processor 210 of the childcare assistance device 20 further includes the childcare record acquisition unit 217 and the childcare record inspection unit 218. The childcare record acquisition unit 217 acquires the childcare record of the infant 2 #1 created by the babysitter 3. The childcare record inspection unit 218 inspects the childcare record acquired by the childcare record acquisition unit 217 by comparing it with the behavior of the infant 2 #1 in the period Tc determined by the determination unit 214. Therefore, in accordance with the childcare assistance device 20, the parent of the infant 2 #1 is capable of evaluating the reliability of the childcare record of the infant 2 #1 created by the babysitter 3.

(Exemplary Modification 7) In the above-described embodiment and the exemplary modifications, the biological information of the infant 2 #1 acquired by the biological information acquisition unit 211 included in the processor 210 of the childcare assistance device 20 is the brain wave data 51 of the infant 2 #1 measured by the brain wave sensor 5. In the present Exemplary Modification 7, the biological information of the infant 2 #1 acquired by the biological information acquisition unit 211 is vocalization data of the infant 2 #1 which is collected by a voice detection device. The voice detection device is used instead of the brain wave sensor 5 constituting the above-described childcare assistance system 1. With respect to an infant 2 #1 who does not like wearing the brain wave sensor 5, it is considered preferable to apply the childcare assistance device 20 in the present Exemplary Modification 7 in which the voice detection device, which does not need to be worn, is used.

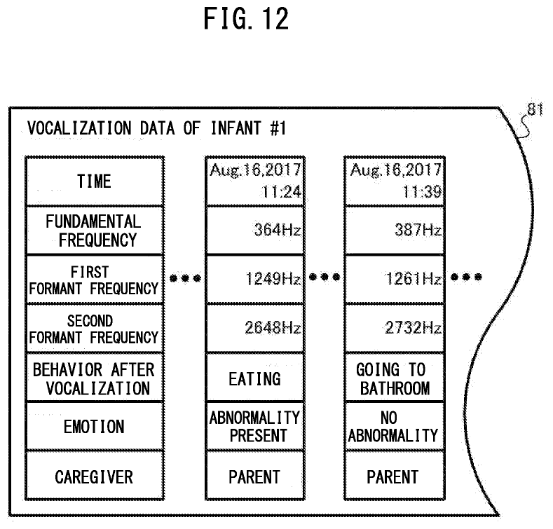

FIG. 12 is a diagram showing vocalization data 81 acquired as biological information of the infant 2 #1 by the childcare assistance device 20 in the Exemplary Modification 7. Similar to the brain wave data 51 in the above-described embodiment and the exemplary modifications, a plurality of frequency components are also contained in the vocalization data 81. The plurality of frequency components included in the vocalization data 81 are, for example, as shown in FIG. 12, a fundamental frequency, a first formant frequency, and a second formant frequency of the vocalizations of the infant 2 #1. Moreover, when a vocalization waveform is approximated by combining a plurality of sine waves, the frequency of the sine wave indicating the lowest frequency from among the plurality of sine waves is referred to as the fundamental frequency. Further, it is known that the first formant frequency and the second formant frequency are obtained in order of closeness from the fundamental frequency as frequencies corresponding to the peak of the amplitude of sine waves indicating frequencies that are integral multiples of the fundamental frequency.

In the vocalization data 81, at each time when the infant 2 #1 makes a vocalization, there are recorded the fundamental frequency, the first formant frequency, and the second formant frequency of such a vocalization, and furthermore, the data is associated with the behavior information, the emotional information, and the caregiver information of the infant 2 #1 immediately after such time, which are acquired by the additional information acquisition unit 215 included in the processor 210 of the childcare assistance device 20. Based on combinations of the values of the plurality of frequency components included in the vocalization data 81, it is possible to estimate the feelings of the infant 2 #1, such as “angry,” “hungry,” “sleepy,” and “wanting to play together.” Therefore, the behavior information indicating the behaviors immediately after the vocalizations of the infant 2 #1 in which these feelings occurred, and the emotional information indicating whether or not an emotional abnormality has occurred in the infant 2 #1 during such behaviors are associated with the data. For example, as shown in FIG. 12, at 11:24 on Aug. 16, 2017, “eating” is associated as the behavior information immediately after the vocalization of the infant 2 #1 in which the feeling of being “hungry” occurred, and in the example shown in FIG. 12, “there is an emotional abnormality” is associated as the emotional information of the infant 2 #1 during such a behavior. At 11:39 on Aug. 16, 2017, “going to the bathroom” is associated as the behavior information immediately after the vocalization of the infant 2 #1 in which the feeling of being “angry” occurred, and in the example shown in FIG. 12, “there is no emotional abnormality” is associated as the emotional information of the infant 2 #1 during such a behavior. Although illustration thereof is omitted in FIG. 12, “sleeping” is associated as the behavior immediately after the vocalization of the infant 2 #1 in which the feeling of being “sleepy” occurred, and the emotional information of the infant 2 #1 during such a behavior is also associated therewith. The behavior of “playing” is associated as the behavior immediately after the vocalization of the infant 2 #1 in which the feeling of “wanting to play together” occurred, and the emotional information of the infant 2 #1 during such a behavior is also associated therewith. In this manner, the behavior information and the emotional information can be associated with the vocalization data 81 of the infant 2 #1.

The additional information acquisition unit 215 acquires the behavior information indicating the behaviors of the infant 2 #1 in the past period Tp1 in which the brain wave data 51 of the infant 2 #1 was measured, together with acquiring the emotional information indicating whether or not an emotional abnormality has occurred in the infant 2 #1 during such behaviors, and associates the behavior information and the emotional information with the vocalization data 81 of the infant 2 #1 in the past period Tp1 acquired by the biological information acquisition unit 211. Using the vocalization data 81 of the infant 2 #1 in the past period Tp1 acquired by the biological information acquisition unit 211 and associated with the behavior information and the emotional information by the additional information acquisition unit 215, and the reference information that is based on the vocalization data 81 of the infant 2 #1 in the past period Tp1 and corresponds to the combination of the type of behavior information and the presence or absence of the occurrence of an emotional abnormality, the biological information estimation unit 213 included in the processor 210 of the childcare assistance device 20 estimates the vocalization data 81 of the infant 2 #1 in the recent or current period Tc that differs from the past period Tp1, for each of the types of combinations of the behavior information and the presence or absence of the occurrence of an emotional abnormality. The determination unit 214 included in the processor 210 of the childcare assistance device 20 determines the behaviors and the emotional abnormalities of the infant 2 #1 in the period Tc, based on the comparison between the actual measured values of the vocalization data 81 of the infant 2 #1 in the period Tc acquired by the biological information acquisition unit 211, and the estimated values of the vocalization data 81 of the infant 2 #1 in the period Tc estimated by the biological information estimation unit 213 for each of combinations of the type of behavior information and the presence or absence of the occurrence of an emotional abnormality.

(Exemplary Modification 8) In the above-described embodiment and the exemplary modifications, the biological information of the infant 2 #1 acquired by the biological information acquisition unit 211 included in the processor 210 of the childcare assistance device 20 includes a plurality of frequency components. The biological information, for example, is the brain wave data 51 or the vocalization data 81. The determination unit 214 included in the processor 210 of the childcare assistance device 20 determines the emotional abnormalities of the infant 2 #1 based on the degree of similarity of a plurality of parameters corresponding to the plurality of frequency components. The plurality of parameters are the percentages of the plurality of frequency components constituting the brain wave data 51, or the values of the plurality of frequency components constituting the vocalization data 81.

However, the above-described biological information may be a combination of a plurality of types of biological information that can be measured from the infant 2 #1. The plurality of types of biological information include, for example, a body temperature, a respiratory rate, a heart rate, a frequency of going to the bathroom, a cerebral blood flow value, and/or an amount of sweating of the infant 2 #1. The determination unit 214 determines an emotional abnormality of the infant 2 #1 based on the degree of similarity of the values of each of these plurality of types of biological information.

The present invention is not limited to the configurations in the above-described embodiments and the various exemplary modifications, insofar as the characteristic functions of the present invention are not impaired.

### REFERENCE SIGNS LIST

1 childcare assistance system
2 infant
3 babysitter
5 brain wave sensor
10 electronic device
11 electronic device
20 childcare assistance device
25 storage device
30 communication network
40 computer program providing server
45 recording medium
50 brain waves
51 brain wave data
52 accumulated information
53 reference information
55 record
56 record
57 record
81 vocalization data
91 record
92 record
96 record
97 record
140 screen
141 notification information
210 processor
211 biological information acquisition unit
212 reference information generating unit
213 biological information estimation unit
214 determination unit
215 additional information acquisition unit
216 notification information generating unit
217 childcare record acquisition unit
218 childcare record inspection unit
220 memory
230 communication module

1. A childcare assistance device, comprising:
a biological information acquisition unit configured to acquire biological information of an infant;
a biological information estimation unit configured to estimate, using reference information based on biological information of a plurality of infants, the biological information of the infant;
a determination unit configured to determine a behavior of the infant on a basis of a comparison between the biological information acquired by the biological information acquisition unit, and the biological information estimated by the biological information estimation unit;
a childcare record acquisition unit configured to acquire a childcare record of the behavior of the infant created by a babysitter who has provided childcare for the infant in a predetermined period; and
a childcare record inspection unit configured to inspect the childcare record by comparing the childcare record with a result of determination of the behavior in the predetermined period as determined by the determination unit.

2. (canceled)

3. (canceled)

4. The childcare assistance device according to claim 1, further comprising a behavior information acquisition unit configured to acquire behavior information indicating the behavior of the infant in a period before the predetermined period, and to associate the behavior information with the biological information in the period before the predetermined period acquired by the biological information acquisition unit, wherein:
the biological information estimation unit estimates the biological information in the predetermined period for each of types of the behavior information, using the biological information in the period before the predetermined period with which the behavior information is associated, and the reference information which corresponds to the types of behavior information of the plurality of infants; and
the determination unit determines the behavior of the infant in the predetermined period, on a basis of the comparison between the biological information in the predetermined period acquired by the biological information acquisition unit, and the biological information in the predetermined period estimated by the biological information estimation unit for each of the types of the behavior information.

5. The childcare assistance device according to claim 4, further comprising an emotional information acquisition unit configured to acquire emotional information indicating whether or not an emotional abnormality has occurred in the infant during the behavior in the period before the predetermined period, and to associate the emotional information with the biological information in the period before the predetermined period acquired by the biological information acquisition unit, wherein:
the biological information estimation unit estimates the biological information in the predetermined period for each of combinations of the type of the behavior information and a presence or absence of the emotional abnormality, using the biological information in the period before the predetermined period with which the behavior information and the emotional information are associated, and the reference information which corresponds to the types of the behavior information and the presence or absence of emotional abnormalities of the plurality of infants; and
the determination unit determines the behavior and the emotional abnormality of the infant in the predetermined period, on a basis of the comparison between the biological information in the predetermined period acquired by the biological information acquisition unit, and the biological information in the predetermined period estimated by the biological information estimation unit for each of the combinations.

6. (canceled)

7. (canceled)

8. (canceled)

9. The childcare assistance device according to claim 5, further comprising a notification information generating unit configured to generate notification information in order to notify, via an electronic device, the babysitter or a manager of the babysitter that the emotional abnormality of the infant has been determined by the determination unit.
